(19) United States
(12) Patent Application Publication
(10) Pub. No.: US 2005/0261550 A1
(43) Pub. Date: Nov. 24, 2005

(54) System, apparatus, and method for supporting insertion of endoscope

(75) Inventors: Shunya Akimoto, Kawasaki-shi (JP); Junichi Onishi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/190,336

(22) Filed: Jul. 27, 2005

(30) Foreign Application Priority Data
Jan. 30, 2004 (JP) 2004-024834

(51) Int. Cl. A61B 1/04

(52) U.S. Cl. 600/117; 600/101; 600/118; 600/109

(57) ABSTRACT

According to the present invention, to guide an endoscope to a target part in vivo with reliability on the basis of guide images corresponding to actual branch points, an endoscope insertion support system includes a VBS image capture unit for capturing VBS images stored in a VBS image storage unit, a navigation VBS video generation unit for generating a navigation VBS video serving as a series of navigation moving pictures to support the insertion of a bronchoscope into a bronchus on the basis of patient information entered from an input device, a navigation VBS video storage unit for storing the navigation VBS video, an image processing unit for performing various image processing operations, and a memory for temporarily storing registered navigation VBS frame images while the navigation VBS video is being generated.

This application claims benefit of Japanese Patent Application No. 2004-24834 filed in Japan on Jan. 30, 2004, the contents of which are incorporated by this reference.

### BACKGROUND OF THE INVENTION

1. Field of the Invention

The present invention relates to a system, apparatus, and method for supporting the insertion of an endoscope.

2. Description of the Related Art

In the medical diagnostic field, diagnoses based on images are coming into wide use. As one of the diagnoses, tomograms of a part of a subject are captured using an x-ray computed tomography (CT) system to obtain three-dimensional (3D) image data of the part. The subject is diagnosed on the basis of the 3D image data of the part. In the CT system, an x-ray generation unit and an x-ray detection unit are continuously rotated around a subject at the same time as the subject is continuously moved along the body axis, thus performing a spiral continuous scan (helical scan) to the subject in three dimensions. 3D images are formed from continuous cross-sectional images (slices) in three dimensions.

The above-mentioned 3D images include a 3D image of a bronchus within lungs. 3D images of bronchi are used to three-dimensionally grasp the position of an abnormal part which may be affected by, e.g., a lung cancer. In this diagnosis, to check the abnormal part by biopsy, a bronchoscope is inserted into a bronchus to collect samples of bronchial tissue using a biopsy needle or biopsy forceps extending from the distal end of the bronchoscope.

With respect to a multi-branching tubular organ, such as a bronchus dividing into smaller bronchi and subsequently bronchioles, if the whole bronchial tree can be displayed on a display screen, it is difficult to confirm and correct the insertion direction of a bronchoscope. Japanese Unexamined Patent Application Publication No. 2000-135215 discloses a navigation system. According to this system, a 3D image of a tubular organ in the body of a subject is produced based on image data of the subject in three dimensions, a path to a target part is obtained along the tubular organ in the 3D image, a virtual endoscopic image of the tubular organ in the path is formed based on the image data, and the virtual endoscopic image is displayed to guide a bronchoscope to the target part.

### SUMMARY OF THE INVENTION

The present invention provides an endoscope insertion support system for guiding an endoscope into a tubular organ in the body of a subject, the tubular organ dividing into branches, the system including: virtual tubular-organ image generating means for generating a plurality of virtual tubular-organ images corresponding to a plurality of insertion points in an insertion path of the tubular organ on the basis of image data in three dimensions in the subject body; start-point and end-point specifying means for specifying a start point and an end point in the insertion path; insertion-direction specifying means for extracting branch points in the insertion path between the start point and the end point to specify the insertion direction of the endoscope in the virtual tubular-organ image corresponding to each extracted branch point; virtual tubular-organ image registering means for registering insertion information regarding the insertion direction specified by the insertion-direction specifying means by corresponding the insertion information to the virtual tubular-organ path image; and insertion-path guide video generating means for generating an insertion-path guide video comprising the virtual tubular-organ images in the insertion path between the start point and the end point via the branch points extracted by the insertion-direction specifying means and the virtual tubular-organ images to which the respective pieces of insertion information registered by the virtual tubular-organ image registering means are added.

The present invention further provides an endoscope insertion support apparatus for guiding an endoscope to a tubular organ in the body of a subject, the tubular organ dividing into branches, the apparatus including: an image generation unit for generating virtual endoscopic frame images of all tubular-organ paths on the basis of tomographic image data of a patient; an image storage unit for storing the virtual endoscopic frame images generated by the image generation unit; an image capture unit for capturing the virtual endoscopic frame images stored in the image storage unit; a navigation virtual endoscopic video generation unit for generating a navigation virtual endoscopic video of the patient on the basis of patient information and the virtual endoscopic frame images captured by the image capture unit and generating branch information regarding branch points in an insertion path, the information being added to the navigation virtual endoscopic video; a navigation virtual endoscopic video storage unit for storing the navigation virtual endoscopic video and the branch information regarding the branch points in the insertion path in such a way that each piece of branch information is linked to the corresponding navigation virtual endoscopic frame image; an image processing unit for processing the navigation virtual endoscopic video and the branch information; and an image display control unit for displaying image data processed by the image processing unit in a monitor.

The present invention further provides an endoscope insertion support method for guiding an endoscope into a tubular organ in the body of a subject, the tubular organ dividing into branches, the method including the steps of: generating virtual endoscopic frame images of tubular-organ paths on the basis of tomograms in the subject body; storing the generated virtual endoscopic frame images; inputting patient information; specifying an insertion support start point and an insertion support end point in a model image of a tubular organ based on the input patient information; capturing the virtual endoscopic frame image corresponding to the insertion support start point and that corresponding to the insertion support end point among the stored virtual endoscopic frame images of the patient on the basis of the input patient information to display the captured virtual endoscopic frame images; temporarily registering in a memory each displayed virtual endoscopic frame image, on which an insertion target marker is superimposed, as a registered frame image to be included in a navigation virtual endoscopic video and further registering positional information of the insertion target marker as branch information regarding a branch point in an insertion path, the temporal registration in the memory being repeated until a registered frame image to be included in the navigation virtual endoscopic video is obtained in a position just before the insertion support end point; and storing the registered frame images corresponding to the desired number stored in the memory and all of the virtual endoscopic frame images in the tubular-organ path, to which the registered frame images corresponding to the desired number are assigned, as the navigation virtual endoscopic video in an image storage unit.

### BRIEF DESCRIPTION OF THE DRAWINGS

FIG. 1 is a block diagram of the structure of a bronchoscope insertion support system according to an embodiment of the present invention;

FIG. 2 is a flowchart explaining the operation of the bronchoscope insertion support system of FIG. 1;

FIG. 3 is a diagram explaining a patient information entry screen displayed in the operation of FIG. 2;

FIG. 4 is a diagram explaining a bronchial-tree model image displayed in the operation of FIG. 2;

FIG. 5 is a first diagram explaining a navigation VBS video set screen displayed in the operation of FIG. 2;

FIG. 6 is a second diagram explaining the navigation VBS video set screen displayed in the operation of FIG. 2;

FIG. 7 is a third diagram explaining the navigation VBS video set screen displayed in the operation of FIG. 2;

FIG. 8 is a fourth diagram explaining the navigation VBS video set screen displayed in the operation of FIG. 2;

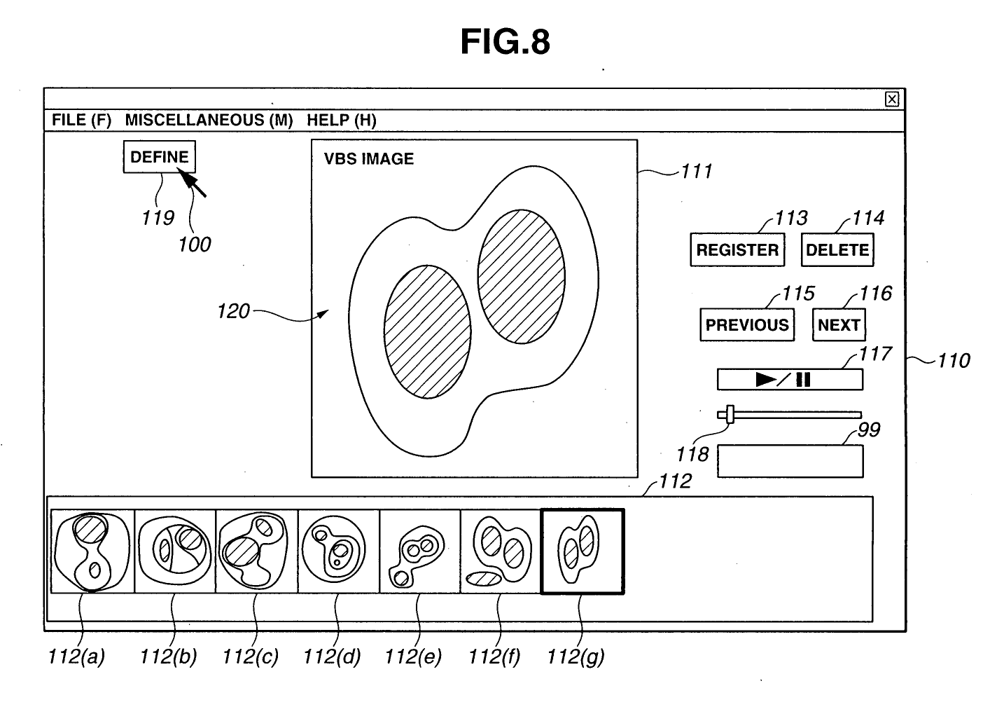

FIG. 9 is a diagram explaining a bronchial-tree model image including a navigation path obtained by the operation of FIG. 2;

FIG. 10 is a fifth diagram explaining the navigation VBS video set screen displayed in the operation of FIG. 2;

FIG. 11 is a sixth diagram explaining the navigation VBS video set screen displayed in the operation of FIG. 2;

FIG. 12 is a seventh diagram explaining the navigation VBS video set screen displayed in the operation of FIG. 2;

FIG. 13 is an eighth diagram explaining the navigation VBS video set screen displayed in the operation of FIG. 2;

FIG. 14 is a block diagram of the structure of a bronchoscope insertion support system according to a first modification of the embodiment in FIG. 1; and

FIG. 15 is a block diagram of the structure of a bronchoscope insertion support system according to a second modification of the embodiment in FIG. 1.

### DETAILED DESCRIPTION OF THE PREFERRED EMBODIMENTS

Embodiments of the present invention will now be described below with reference to the drawings.

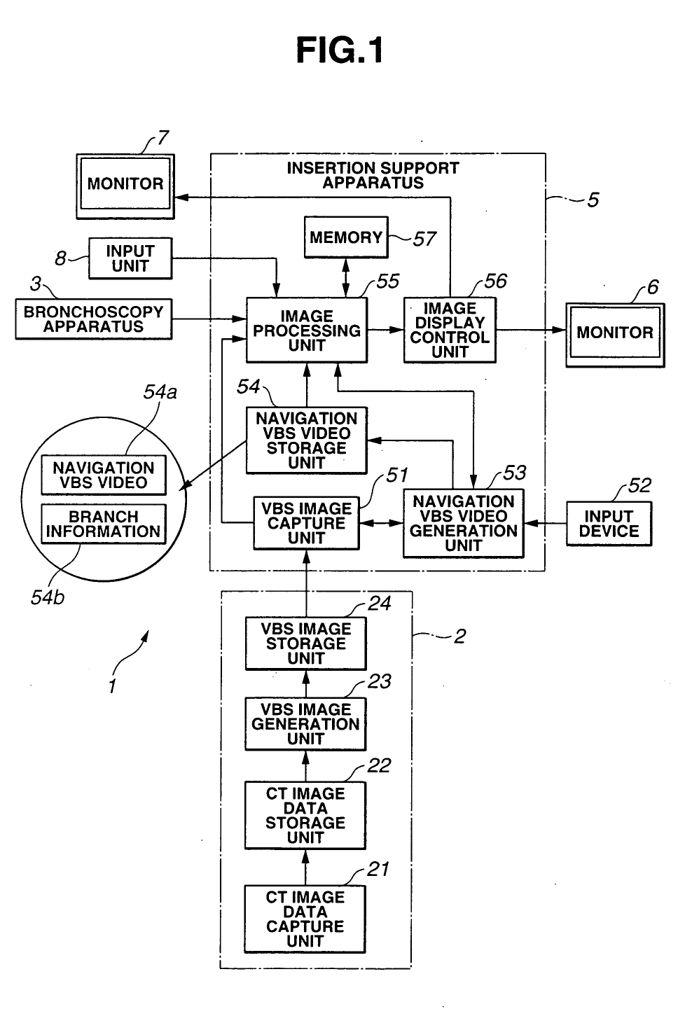

FIG. 1 shows a system 1 for supporting the insertion of an endoscope (bronchoscope) into a bronchus according to an embodiment of the present invention. The system 1 includes a VBS image generation apparatus 2 for generating a virtual endoscopic image of inside of bronchus according to a virtual bronchoscopy system (hereinafter, referred to as a VBS image), a bronchoscopy apparatus 3, and an insertion support apparatus 5. The VBS image generation apparatus 2 generates a VBS image based on CT image data. The insertion support apparatus 5 combines an endoscopic image (hereinafter, referred to as a live image) captured by the bronchoscopy apparatus 3 with the VBS image obtained by the VBS image generation apparatus 2 and displays the combined image in monitors 6 and 7 so as to support the insertion of the bronchoscopy apparatus 3 into a bronchus.

The bronchoscopy apparatus 3 includes a bronchoscope having image pickup means, a light source for supplying an illumination beam to the bronchoscope, and a camera control unit for processing image pickup signals supplied from the bronchoscope. The components of the apparatus 3 are not shown in the diagram. The bronchoscopy apparatus 3 allows the bronchoscope inserted in a bronchus in the body of a patient to capture an image of the surface of a bronchus and biopsy an affected part at the end of a bronchus, combines a live image with a VBS image, and displays the combined image in the monitor 7. An input unit 8 including a pointing device, such as a touch panel, is provided for the monitor 7. While an operator inserts the bronchoscope into the body of the patient and operates it, a nurse, serving as an assistant, can easily operate the input unit 8 including the touch panel in accordance with an instruction of the operator.

The VBS image generation apparatus 2 includes a CT image data capture unit 21, a CT image data storage unit 22, a VBS image generation unit 23, and a VBS image storage unit 24. The CT image data capture unit 21 captures CT image data, serving as three-dimensional (3D) image data, which is generated by a known CT apparatus (not shown) for capturing tomograms of the patient, through a removable storage medium, such as a magnetic optical (MO) disk or a digital versatile disk (DVD). The CT image data storage unit 22 stores the CT image data captured by the CT image data capture unit 21. The VBS image generation unit 23 generates VBS images as frame images of all paths in the bronchial tree of the patient on the basis of the CT image data stored in the CT image data storage unit 22. The VBS image storage unit 24 stores the VBS images generated by the VBS image generation unit 23.

The insertion support apparatus 5 includes a VBS image capture unit 51, a navigation VBS video generation unit 53, a navigation VBS video storage unit 54, an image processing unit 55, an image display control unit 56, and a memory 57. The VBS image capture unit 51 captures the VBS images stored in the VBS image storage unit 24. The navigation VBS video generation unit 53 generates a navigation VBS video serving as a series of navigation moving pictures used to support the insertion of the bronchoscope into a bronchus on the basis of input information (patient information) supplied from an input device 52. In addition, the navigation VBS video generation unit 53 generates branch information regarding a branch point (e.g., a bifurcation), the branch information being added to the navigation VBS video. The navigation VBS video storage unit 54 stores the navigation VBS video and the branch information as data files 54a and 54b in such a way that the navigation VBS video is linked to the branch information. The image processing unit 55 performs various image processing operations. The image display control unit 56 displays processed image data in the monitor 6. The memory 57 temporarily stores a registered navigation VBS frame image while the navigation VBS video is being generated.

The image processing unit 55 generates a navigation VBS video set image (screen) used to generate the navigation VBS video through the navigation VBS video generation unit 53 on the basis of the VBS images captured by the VBS image capture unit 51. In addition, the image processing unit 55 generates an insertion support image (screen) having a multi-window showing a navigation VBS frame image, on which the branch information is superimposed, and a live image. The image processing unit 55 displays the screens in the monitors 6 and 7.

The operation of the system according to the present embodiment will now be described below.

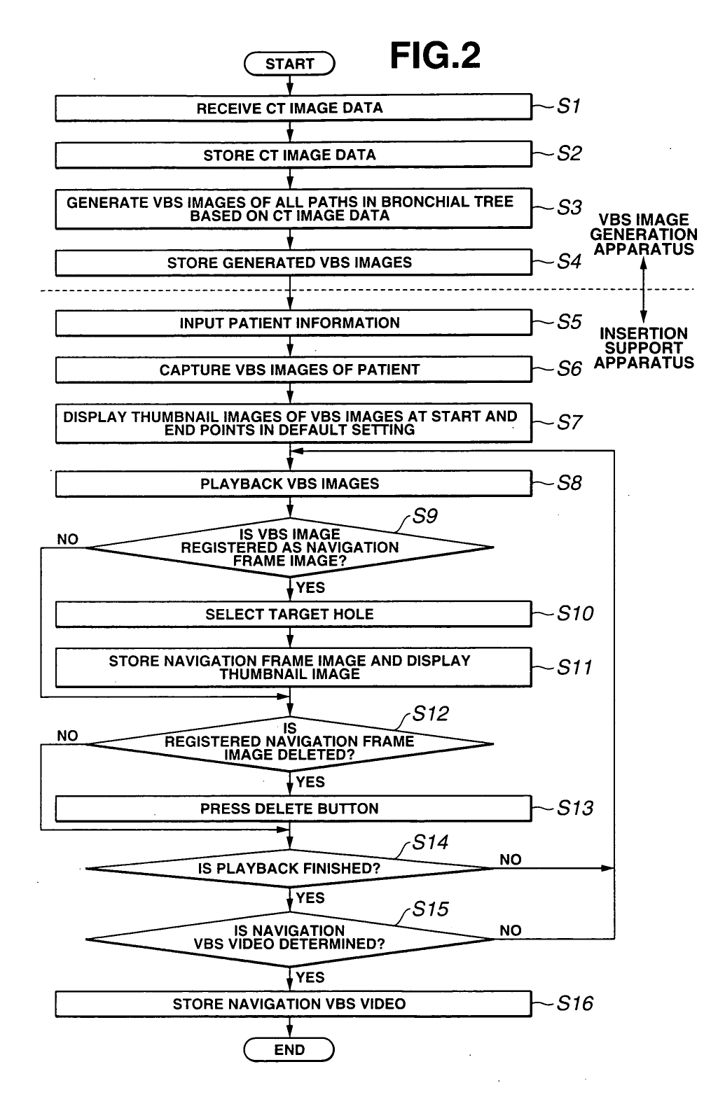

Referring to FIG. 2, in step S1, the VBS image generation apparatus 2 receives CT image data through the CT image data capture unit 21. In step S2, the CT image data storage unit 22 stores the CT image data. In step S3, the VBS image generation unit 23 generates VBS images as frame images of all paths in the bronchial tree of the patient on the basis of the CT image data stored in the CT image data storage unit 22. In step S4, the VBS image storage unit 24 stores the VBS images generated by the VBS image generation unit 23. Thus, a VBS image generation process by the VBS image generation apparatus 2 is terminated. Step S5 and subsequent steps are executed in the insertion support apparatus 5.

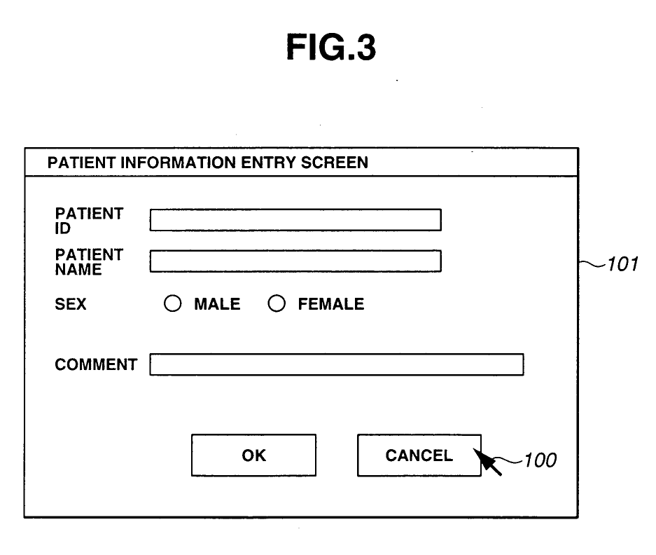

After the VBS image generation process by the VBS image generation apparatus 2 is terminated, in step S5, the image processing unit 55 of the insertion support apparatus 5 displays a patient information entry screen 101, as shown in FIG. 3, in the monitor 6 and enters a standby mode until patient information (patient ID, patient name, sex, or a comment) in each field specified by a pointer 100 is entered by the input device 52.

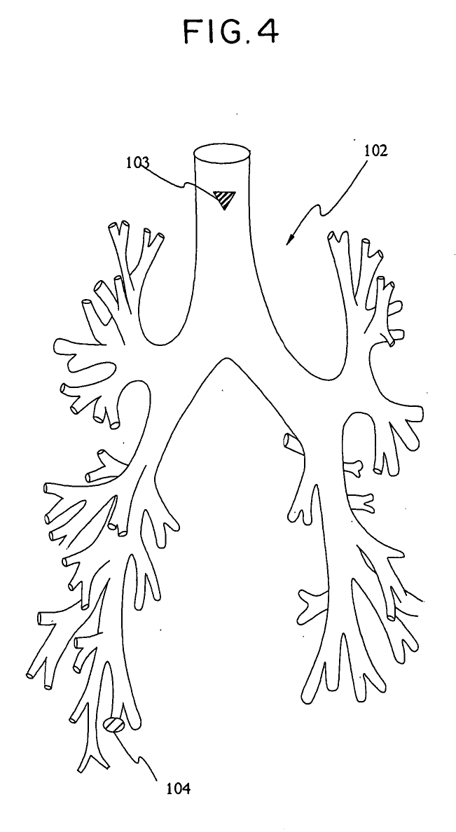

After the patient information is entered in the patient information entry screen 101, the image processing unit 55 displays a bronchial-tree model image 102 in the monitor 6 as shown in FIG. 4. In the model image 102, an insertion support start point 103 and an insertion support end point 104, which serves as an area of interest such as an affected part, are specified using the pointer 100.

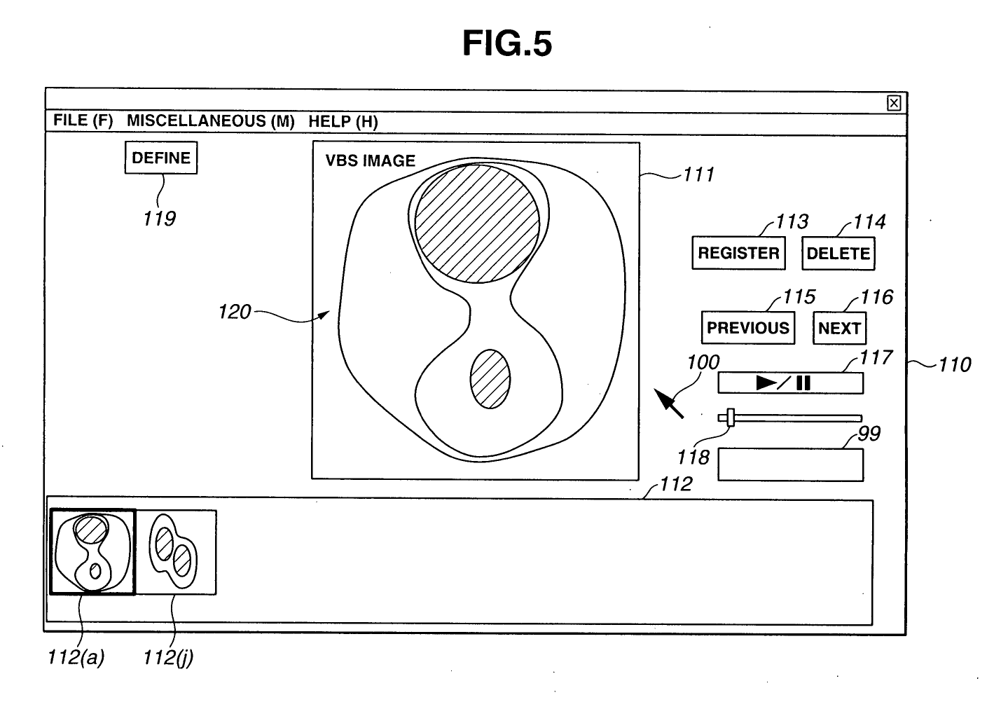

After the patient information, the insertion support start point 103, and the insertion support end point 104 are input, in step S6, the image processing unit 55 captures a VBS image corresponding to the insertion support start point 103 and that corresponding to the insertion support end point 104 of the VBS images of all paths in the bronchial tree of the corresponding patient through the VBS image capture unit 51. In step S7, the image processing unit 55 displays a navigation VBS video set screen 110, as shown in FIG. 5, in the monitor 6.

Referring to FIG. 5, the navigation VBS video set screen 110 includes a VBS image display area 111, a thumbnail image display area 112, and an error/comment display area 99. The VBS image display area 111 displays a VBS image 120 corresponding to the insertion support start point 103 in full-screen mode. The thumbnail image display area 112 displays a thumbnail image of the VBS image 120.

If an error occurs, the error/comment display area 99 displays an error message, thus informing a user of the occurrence of the error. In addition, the error/comment display area 99 can display a comment.

In FIG. 5, the thumbnail image display area 112 shows a thumbnail image 112a of the VBS image 120 corresponding to the insertion support start point 103 and a thumbnail image 112j of a VBS image 120 corresponding to the insertion support end point 104. As will be described hereinafter, the thumbnail image display area 112 further displays thumbnail images of VBS images 120, serving as registered navigation VBS frame images corresponding to some points in a path in the bronchial tree in addition to the above thumbnail images.

When the thumbnail image display area 112 includes a thumbnail image which matches a VBS image 120 displayed in the VBS image display area 111, the matching thumbnail image is framed by a bold line so that the relationship between the VBS image 120 in the VBS image display area 111 and the thumbnail image in the thumbnail image display area 112 can be easily understood. In FIG. 5, the VBS image 120 in the VBS image display area 111 corresponds to that of the insertion support start point 103. Accordingly, the frame of the thumbnail image 112a is shown by the bold line in the thumbnail image display area 112.

The navigation VBS video set screen 110 includes a register button 113, a delete button 114, a previous button 115, a next button 116, a play/stop button 117, a speed designation bar 118, and a define button 119. The register button 113 is used to register a navigation VBS frame image. The delete button 114 is used to delete a registered navigation VBS frame image. The previous button 115 is used to skip to the previous frame image of the registered navigation VBS frame image and the next button 116 is used to skip to the next frame image thereof. The play/stop button 117 is used to play moving pictures in the VBS image display area 111 or stop the playback. The speed designation bar 118 is used to designate playback speed of moving pictures in the VBS image display area 111. The define button 119 is used to define a navigation VBS video serving as a series of moving pictures obtained when a series of navigation VBS frame images is registered, thus storing the navigation VBS video as a moving picture file in the navigation VBS video storage unit 54.

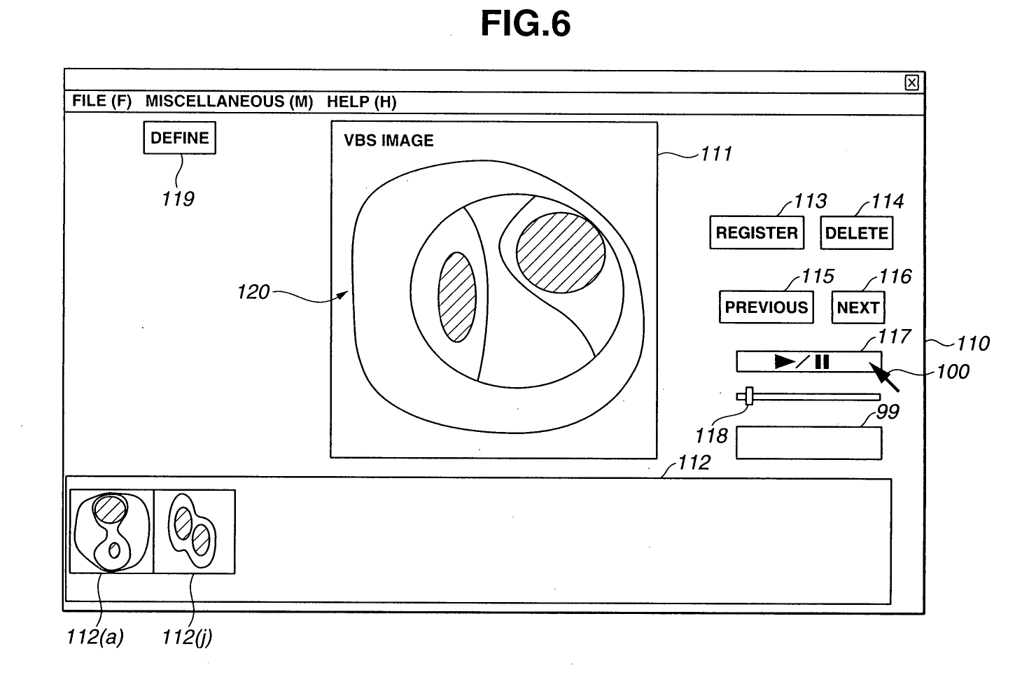

Again referring to FIG. 2, in step S8, the play/stop button 117 is pressed using the pointer 100 as shown in FIG. 6, thus starting the playback of a series of VBS images as moving pictures from the VBS image corresponding to the insertion support start point 103 in the VBS image display area 111.

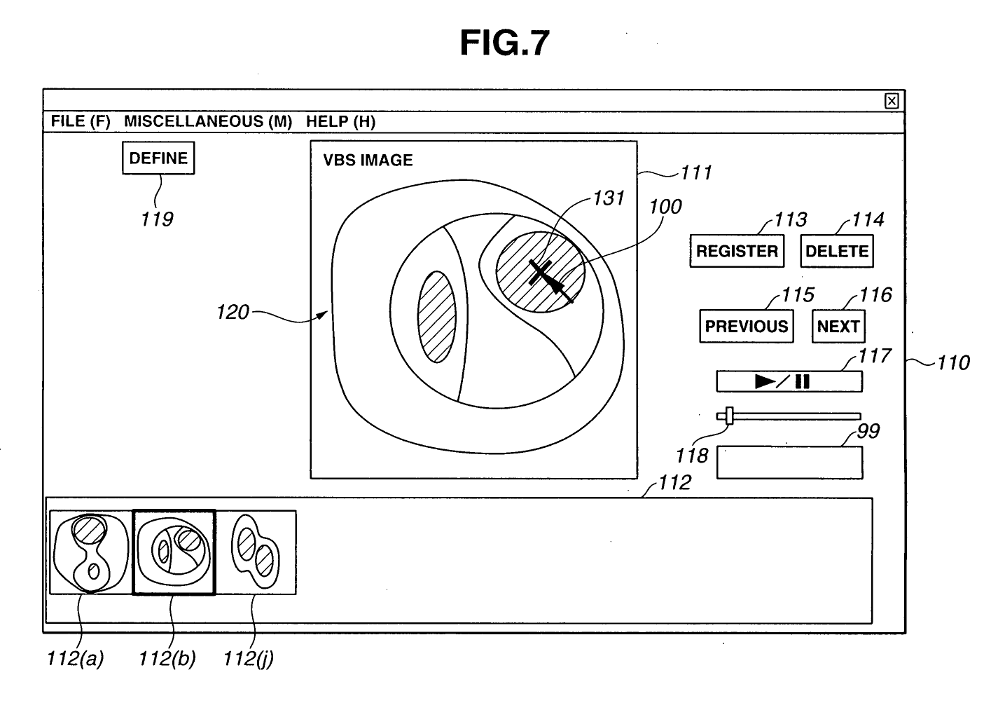

In step S9, when the play/stop button 117 is pressed using the pointer 100 to stop the playback of VBS images as shown in FIG. 6, the process enters a register mode, so that it is determined that registering a navigation VBS frame image is instructed. In step S10, an insertion target to which the bronchoscope will be inserted is selected using the pointer 100 in the VBS image, serving as a still frame image, in the VBS image display area 111. Consequently, as shown in FIG. 7, a target marker 131 is superimposed on the selected hole in the VBS image using the pointer 100. The register button 113 is pressed using the pointer 100, so that the VBS image is temporarily stored as a registered navigation VBS frame image in the memory 57 in step S11. At that time, positional information of the target marker 131 is stored as branch information in the memory 57 in addition to the registered navigation VBS frame image. A thumbnail image 112b of the registered navigation VBS frame image is displayed in the thumbnail image display area 112. The process proceeds to step S12. If the play/stop button 117 is not pressed using the pointer 100 in step S9, the process skips to step S12.

The thumbnail image 112b corresponds to a position between the insertion support start point 103 and the insertion support end point 104. Therefore, the thumbnail image 112b is displayed between the thumbnail images 112a and 112j.

In steps S12 and S13, a thumbnail image to be deleted is selected and is then deleted by pressing the delete button 114 using the pointer 100. The deletion will be described in detail hereinafter.

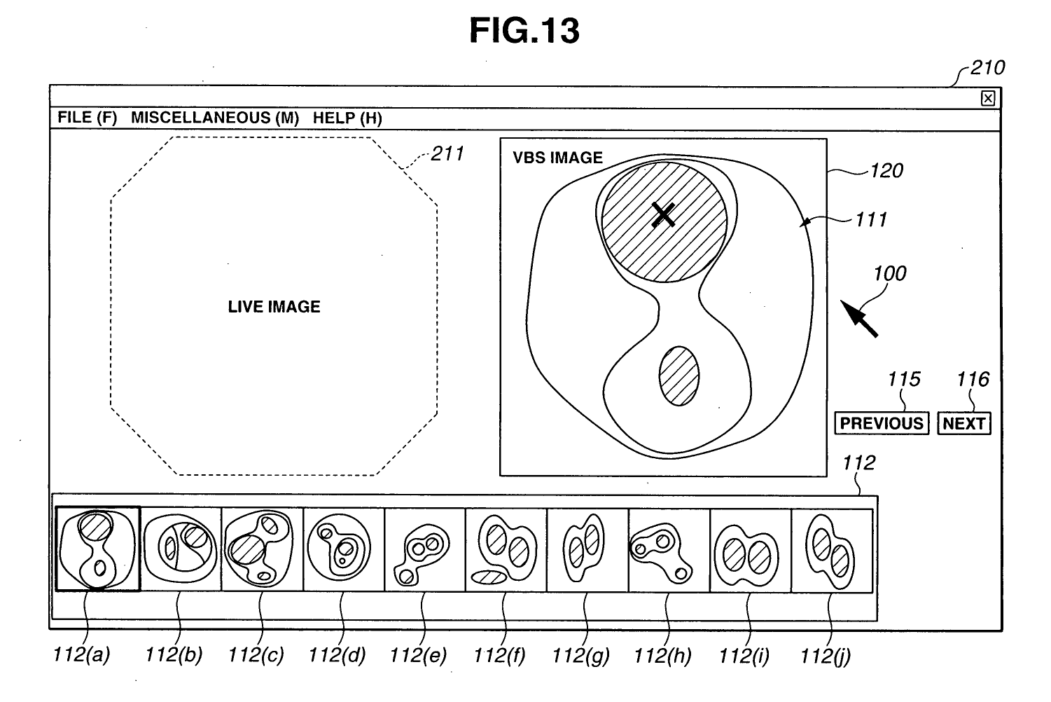

Steps S8 to S13 are repeated until registered navigation VBS frame images corresponding to desired positions up to the insertion support end point 104 are obtained in step S14. As shown in FIG. 13, thumbnail images 112b to 112i of registered navigation VBS frame images corresponding to the desired number are displayed between the thumbnail images 112a and 112j in the thumbnail image display area 112. In each of the registered navigation VBS frame images corresponding to the thumbnail images 112b to 112i, a support target can be designated by the target marker 131.

In the above-mentioned case where the thumbnail images 112b to 112i of the registered navigation VBS frame images corresponding to the desired number are displayed between the thumbnail images 112a and 112j, in step S15, when the define button 119 is pressed using the pointer 100, it is determined that a navigation VBS video is defined. In step S16, the registered navigation VBS frame images corresponding to the desired number stored in the memory 57 and all of VBS images in the bronchial-tree path, to which the registered navigation VBS frame images corresponding to the desired number are assigned, are stored as a navigation VBS video, serving as a moving picture file, in the navigation VBS video storage unit 54. The process is terminated. If the navigation VBS video is not defined, steps S8 to S15 are repeated. The branch information and the navigation VBS video are stored in the navigation VBS video storage unit 54 in such a way that each piece of branch information is linked to the corresponding navigation VBS frame image.

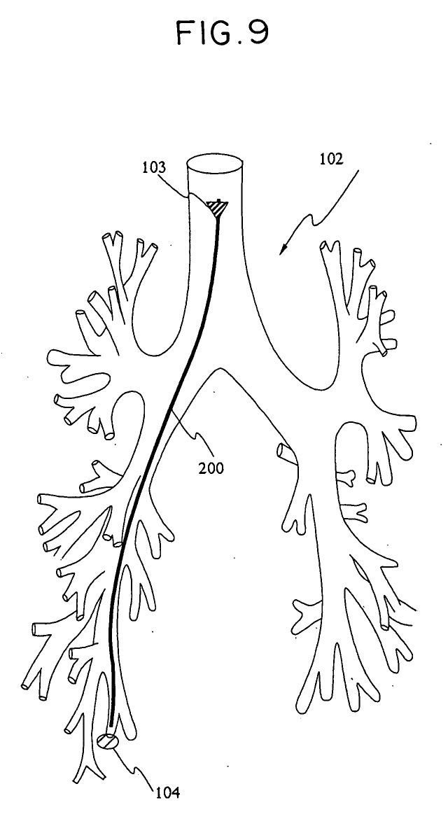

As mentioned above, after the registered navigation VBS frame images corresponding to the desired number are determined, a bronchial-tree path 200 to which the desired registered navigation VBS frame images are assigned is determined as shown in FIG. 9. The navigation VBS video storage unit 54 stores a navigation VBS video, including the registered navigation VBS frame images assigned to the bronchial-tree path 200, as a moving picture file and also stores branch information, each piece of branch information being linked to the corresponding navigation VBS frame image.

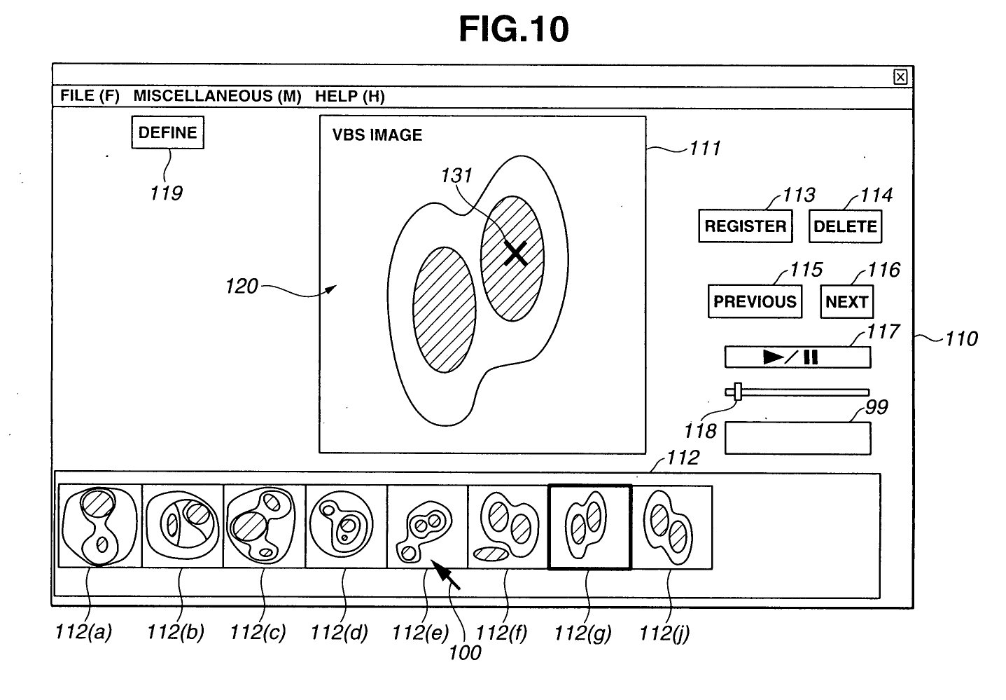

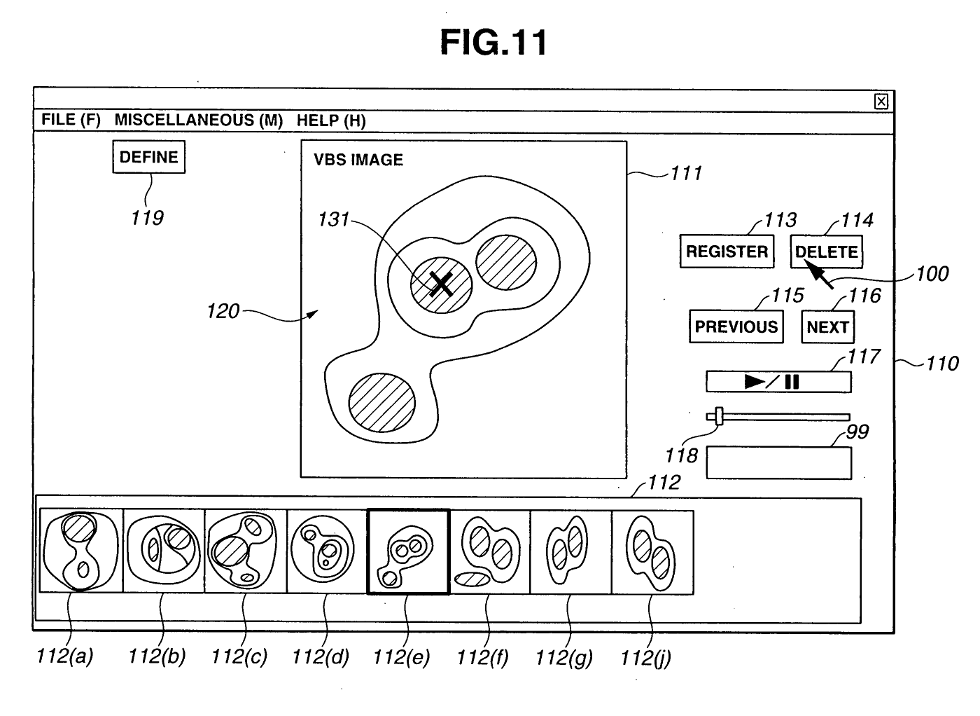

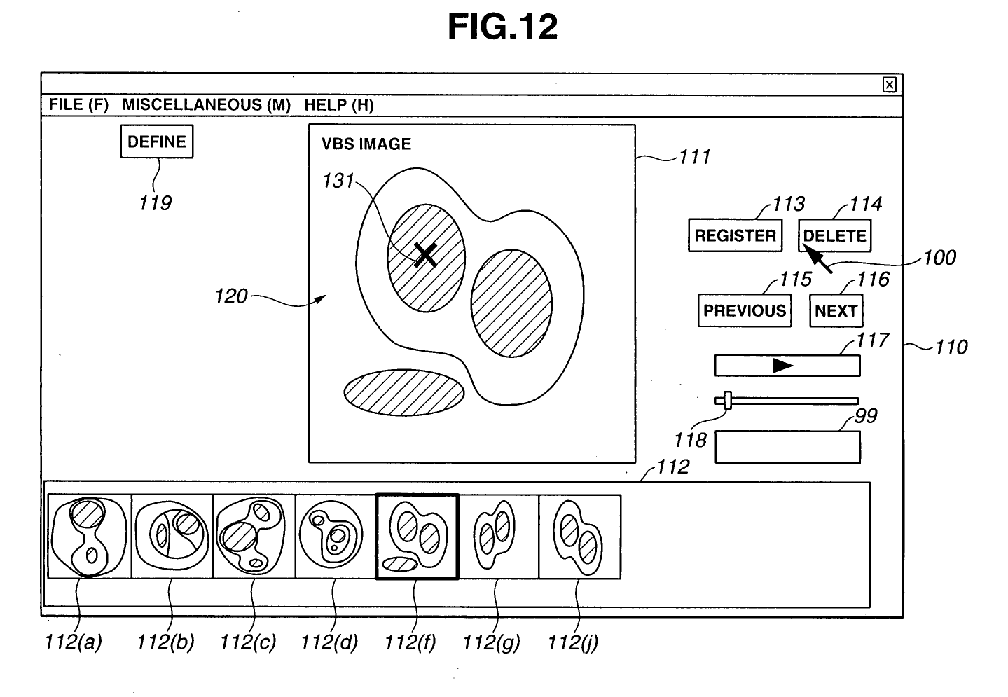

The deletion in steps S12 and S13 of FIG. 2 will now be described below. Referring to FIG. 10, assuming that the operator intends to delete a registered navigation VBS frame image corresponding to, e.g., the thumbnail image 112e after the registered navigation VBS frame images corresponding to the predetermined number are stored in the memory 57, in step S12, the thumbnail image 112e is selected using the pointer 100, so that the thumbnail image 112e is framed by the bold line as shown in FIG. 11. Simultaneously, the registered navigation VBS frame image corresponding to the thumbnail image 112e is displayed in the VBS image display area 111. When the delete button 114 is pressed using the pointer 100 in step S13, the registered navigation VBS frame image corresponding to the thumbnail image 112e is deleted as shown in FIG. 12. Thus, the thumbnail image 112e is deleted in the thumbnail image display area 112. Consequently, for example, the next thumbnail image 112f is framed by the bold line and the registered navigation VBS frame image corresponding to the thumbnail image 112f is displayed in the VBS image display area 111. The deletion is executed in this manner.

As mentioned above, the navigation VBS video including the registered navigation VBS frame images assigned to the bronchial-tree path 200 is stored as a series of frame images, i.e., as a moving picture file. The insertion support apparatus 5 supports the insertion of the bronchoscope included in the bronchoscopy apparatus 3 into a bronchus using the navigation VBS video.

Specifically, an insertion support screen 210 as shown in FIG. 13 is displayed in the monitor 6. The insertion support screen 210 includes a live image area 211 to display a live (endoscopic) image generated by the bronchoscopy apparatus 3 in addition to the VBS image display area 111 and the thumbnail image display area 112.

The insertion support screen 210 further includes the previous button 115 and the next button 116 to skip to the previous or the next frame image of the registered navigation VBS frame image.

In the insertion support screen 210, a live image is displayed in the live image area 211 and any thumbnail image selected in the thumbnail image display area 112 is displayed as a navigation VBS frame image in the VBS image display area 111. The target marker 131 can be shown in the navigation VBS frame image. The operator finds an insertion hole designated by the target marker 131 in the live image and controls the insertion operation. Consequently, the operator can easily insert the bronchoscope into a bronchus and move it up to an area of interest, such as an affected part, at the insertion support end point 104 through the proper path 200 with reliability.

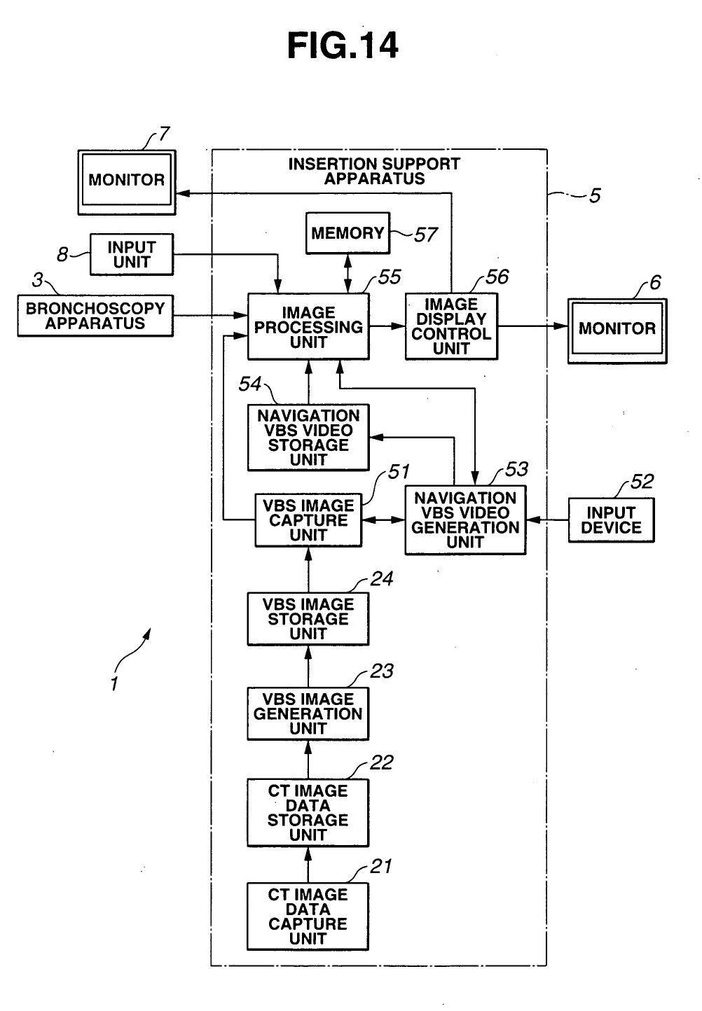

According to the present embodiment, the VBS image generation apparatus 2 is separated from the insertion support apparatus 5. The VBS image generation apparatus 2 includes the CT image data capture unit 21, the CT image data storage unit 22, the VBS image generation unit 23, and the VBS image storage unit 24. The structure of the system is not limited to the above. As shown in FIG. 14, the insertion support apparatus 5 can include the CT image data capture unit 21, the CT image data storage unit 22, the VBS image generation unit 23, and the VBS image storage unit 24.

According to the present embodiment, as described with reference to FIG. 4, the insertion support start point 103 and the insertion support end point 104 are specified. When a target insertion hole is determined at a branch point following the insertion support start point 103, a VBS image is displayed so that the operator can select a target insertion hole (using the target marker 131) and register a navigation VBS frame image. Thus, the final path 200 can be determined as shown in FIG. 9. The operation is not limited to the above. After the insertion support start point 103 and the insertion support end point 104 are specified, the path 200 from the insertion support start point 103 to the insertion support end point 104 is automatically calculated, insertion holes are selected (using the target markers 131) in the automatically calculated path 200. A navigation VBS video including pieces of branch information and registered navigation VBS frame images can be registered in such a way that each piece of branch information is linked to the corresponding navigation VBS frame image.

In the use of the automatically calculated path 200, before an insertion target is determined (using the target marker 131), a recommended target marker is automatically generated as recommended branch information suited for an insertion target, and a VBS image with the recommended target marker is generated and displayed. In the selection, the recommended target marker is shown and, if necessary, is corrected to obtain branch information which is used for actual navigation. A navigation VBS video including pieces of branch information and registered navigation VBS frame images can be registered in such a way that each piece of branch information is linked to the corresponding registered navigation VBS frame image.

In the use of the automatically calculated path 200, for example, the navigation VBS video generation unit 53 in FIG. 1 or 14 calculates a path 200 based on the insertion support start point 103 and the insertion support end point 104 and then captures VBS images, which are assigned to the path 200, stored in the VBS image storage unit 24 through the VBS image capture unit 51. In addition, the navigation VBS video generation unit 53 automatically generates the above-mentioned recommended branch information and generates and displays VBS images to which the recommended branch information is added.

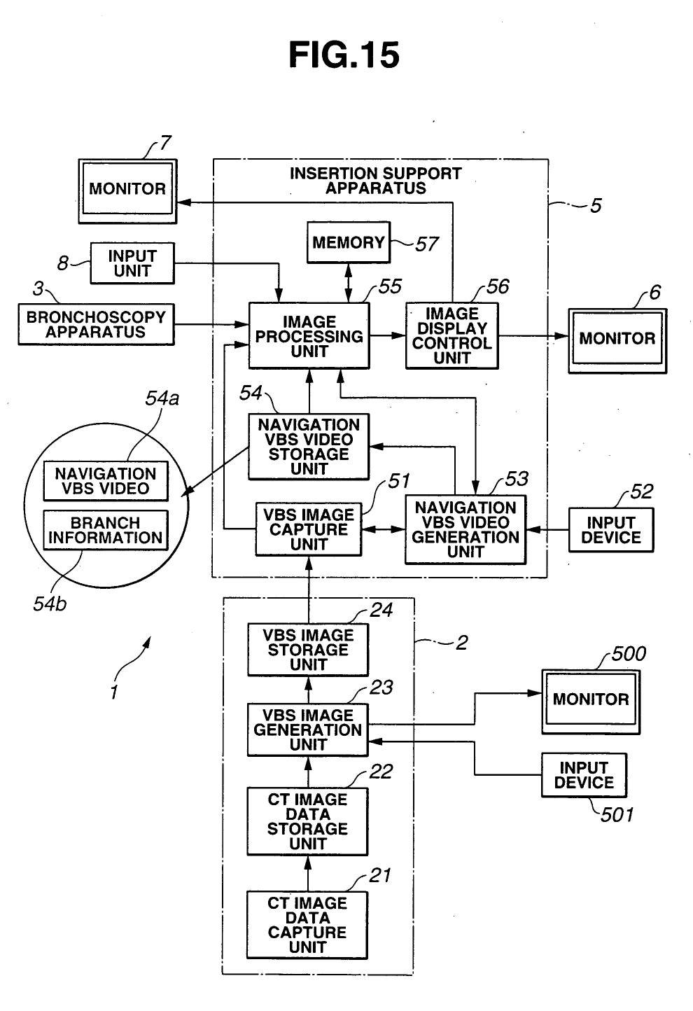

With regard to the automatic calculation of the path 200, instead of the navigation VBS video generation unit 53, as shown in FIG. 15, the VBS image generation unit 23 can execute the calculation on condition that the VBS image generation apparatus 2 includes a monitor 500 and an input device 501. In this instance, the VBS image generation unit 23 can automatically generate a recommended target marker mentioned above. Instead of the bronchial-tree model image 102 in FIG. 4, the VBS image generation unit 23 can specify the insertion support start point 103 and the insertion support end point 104 using multiplanar reconstruction images (MPR images: coronal image, axial image, and sagittal image) generated on the basis of CT image data.

In the present invention, it will be apparent that a wide range of different embodiments can be formed based on this invention without departing from the spirit and scope of this invention. This invention will be restricted by the appended claims but not be limited to any particular embodiment.

1. An endoscope insertion support system for guiding an endoscope into a tubular organ in the body of a subject, the tubular organ dividing into branches, the system comprising:
virtual tubular-organ image generating means for. generating a plurality of virtual tubular-organ images corresponding to a plurality of insertion points in an insertion path of the tubular organ on the basis of image data in three dimensions in the subject body;
start-point and end-point specifying means for specifying a start point and an end point in the insertion path;
insertion-direction specifying means for extracting branch points in the insertion path between the start point and the end point to specify the insertion direction of the endoscope in the virtual tubular-organ image corresponding to each extracted branch point;
virtual tubular-organ image registering means for registering insertion information regarding the insertion direction specified by the insertion-direction specifying means by corresponding the insertion information to the virtual tubular-organ image; and
insertion-path guide video generating means for generating an insertion-path guide video comprising the virtual tubular-organ images in the insertion path between the start point and the end point via the branch points extracted by the insertion-direction specifying means and the virtual tubular-organ images to which the respective pieces of insertion information registered by the virtual tubular-organ image registering means are added.

2. The system according to claim 1, wherein image data in three dimensions in the subject body is generated every frame of all paths in the bronchial tree on the basis of tomographic image data of a patient.

3. The system according to claim 1, further comprising:
virtual tubular-organ image deleting means for deleting a virtual tubular-organ image added to the corresponding insertion information registered by the virtual tubular-organ image registering means.

4. The system according to claim 1, further comprising:
image display control means for displaying a virtual tubular-organ image, corresponding to each branch point, included in the insertion-path guide video generated by the insertion-path guide image generating means as a thumbnail image in a monitor.

5. An endoscope insertion support apparatus for guiding an endoscope to a tubular organ in the body of a subject, the tubular organ dividing into branches, the apparatus comprising:
an image generation unit for generating virtual endoscopic frame images of all tubular-organ paths on the basis of tomographic image data of a patient;
an image storage unit for storing the virtual endoscopic frame images generated by the image generation unit;
an image capture unit for capturing the virtual endoscopic frame images stored in the image storage unit;
a navigation virtual endoscopic video generation unit for generating a navigation virtual endoscopic video of the patient on the basis of patient information and the virtual endoscopic frame images captured by the image capture unit and generating branch information regarding branch points in an insertion path, the information being added to the navigation virtual endoscopic video;
a navigation virtual endoscopic video storage unit for storing the navigation virtual endoscopic video and the branch information regarding the branch points in the insertion path in such a way that each piece of branch information is linked to the corresponding navigation virtual endoscopic frame image;
an image processing unit for processing the navigation virtual endoscopic video and the branch information; and
an image display control unit for displaying image data processed by the image processing unit in a monitor.

6. The apparatus according to claim 5, further comprising:
a memory for temporarily storing a registered navigation virtual endoscopic frame image while the navigation virtual endoscopic video is being generated.

7. The apparatus according to claim 5, wherein the image processing unit superimposes the branch information regarding the branch points in the insertion path on the navigation virtual endoscopic video on the basis of the virtual endoscopic frame images captured by the image capture unit.

8. The apparatus according to claim 5, wherein the image display control unit displays each piece of branch information processed by the image processing unit as a thumbnail image in the monitor.

9. An endoscope insertion support method for guiding an endoscope into a tubular organ in the body of a subject, the tubular organ dividing into branches, the method comprising the steps of:
generating virtual endoscopic frame images of tubular-organ paths on the basis of tomograms in the subject body;
storing the generated virtual endoscopic frame images;
inputting patient information;
specifying an insertion support start point and an insertion support end point in a model image of a tubular organ based on the input patient information;
capturing the virtual endoscopic frame image corresponding to the insertion support start point and that corresponding to the insertion support end point among the stored virtual endoscopic frame images of the patient on the basis of the input patient information to display the captured virtual endoscopic frame images in the monitor;
temporarily registering in a memory each displayed virtual endoscopic frame image, on which an insertion target marker is superimposed, as a registered frame image to be included in a navigation virtual endoscopic video and further registering positional information of the insertion target marker as branch information regarding a branch point in an insertion path, the temporal registration in the memory being repeated until a registered frame image to be included in the navigation virtual endoscopic video is obtained in a position just before the insertion support end point; and
storing the registered frame images corresponding to the desired number stored in the memory and all of the virtual endoscopic frame images in the tubular-organ path, to which the registered frame images corresponding to the desired number are assigned, as the navigation virtual endoscopic video in an image storage unit.

10. The method according to claim 9, further comprising a step of:
deleting a registered frame image to be included in the navigation virtual endoscopic video temporarily registered in the memory, the branch information regarding a branch point in the insertion path being superimposed on the registered frame image.
